(19) United States
(12) Patent Application Publication
(10) Pub. No.: US 2026/0098865 A1
(43) Pub. Date: Apr. 9, 2026

(54) Method for Evaluating Sample Containing Virus Particles, Device for Evaluating Sample Containing Virus Particles and Program for Evaluating Sample Containing Virus Particles

(71) Applicants: SHIMADZU CORPORATION, Kyoto-shi (JP); The University of Osaka, Osaka (JP)

(72) Inventors: Susumu UCHIYAMA, Osaka (JP); Yuki YAMAGUCHI, Osaka (JP); Ryoji NAKATSUKA, Kyoto-shi (JP)

(73) Assignees: SHIMADZU CORPORATION, Kyoto-shi (JP); The University of Osaka, Osaka (JP)

(21) Appl. No.: 19/346,230

(22) Filed: Sep. 30, 2025

(30) Foreign Application Priority Data
Oct. 3, 2024 (JP) 2024-174495

(51) Int. Cl. G01N 33/68; H01J 49/00

(52) U.S. Cl. CPC G01N 33/6851; H01J 49/0036; G01N 2333/015

(57) ABSTRACT

A method for evaluating a sample containing virus particles includes the steps of: preparing a distribution of a detection intensity of a capsid of virus particles detected for each mass of the capsid, the distribution obtained by an analysis of predetermined virus particles contained in one or more samples and having a capsid composed of proteins having known masses; determining an approximate function expressing the distribution of the detection intensity, including the mass as an explanatory variable; and calculating, for a specified mass which is one of the masses at which a response variable of the approximate function has a predetermined value, the number of each protein forming the capsid having the specified mass, based on the specified mass, the mass of each protein forming the capsid, and a subunit number which is the total number of the proteins forming the capsid of the predetermined virus particles.

### TECHNICAL FIELD

The present invention relates to a method for evaluating a sample containing virus particles, a device for evaluating a sample containing virus particles, and a program for evaluating a sample containing virus particles.

### BACKGROUND ART

Gene therapy has been known as a type of method for treating genetic diseases, such as hemophilia, or refractory diseases, such as cancer. In gene therapy, a normal gene is introduced into the cells of a patient who suffers from a gene disease due to the lack of that normal gene, or a tumor-suppressor gene is introduced into the cells of a cancer patient, in order to fundamentally cure those diseases. There are various methods for introducing a gene into cells of a patient, of which a virus vector is often used in order to efficiently introduce a gene into cells of a patient. This technique utilizes the nature of virus particles where a virus particle which has adsorbed to a cell sends its own genome into that cell. Based on this fact, a virus particle carrying a gene for the treatment (“virus vector”) is administered to a patient as a genetic medicine to introduce the gene for the treatment into the cells of the patient. Typical examples of virus vectors are an adenovirus vector, adeno-associated virus (AAV) vector, lentivirus vector and retrovirus vector.

In general, the capsid of a virus particle is a polymer composed of one or more kinds of proteins. Those proteins characterize the infectivity and other natures of the virus particle. Take the example of AAV mentioned earlier: The capsid of a virus particle of AAV is typically considered to be a hexacontamer formed by three kinds of virus proteins VP1, VP2 and VP3 bonded in an approximate ratio of 5:5:50. VP1 affects the transduction efficiency and the infectivity of AAV, while VP2 and VP3 contribute to the creation of the envelope.

In the case of using a virus vector to introduce genes, a huge amount of virus vector is necessary in order to enhance the genetic expression level. However, the virus particles of common virus vectors, including AAV, often differ from each other in the stoichiometric proportion (composition ratio) of the proteins forming the capsid even when those virus particles are of the same kind. This means that they may differ from each other in infectivity and other natures. Therefore, for a gene therapy using a virus vector, the stoichiometric proportion of the proteins forming the capsid of the virus particle which will act as the virus vector needs to be previously known as a critical quality attribute (CQA) for each production lot (culture medium), for example.

### CITATION LIST

### Patent Literature

Patent Literature 1: U.S. Pat. No. 10,585,103 B

### Non Patent Literature

Non Patent Literature 1: Oyama, Hiroaki, et al., “Characterization of adeno-associated virus capsid proteins with two types of VP3-related components by capillary gel electrophoresis and mass spectrometry”, Human gene therapy 32.21-22 (2021): 1403-1416.

### SUMMARY OF INVENTION

### Technical Problem

Non Patent Literature 1 describes methods for identifying and quantifying proteins forming the capsid of a virus particle of AAV using capillary gel electrophoresis and liquid chromatograph mass spectrometry (LC/MS). In these analytical techniques, virus particles are broken into fragments and an analysis is performed in units of proteins or peptides forming the capsid of the virus particle. Although the total-amount ratio of the proteins contained in the sample subjected to the analysis can thereby be determined, the stoichiometric proportion of the proteins forming the capsid of each virus particle cannot be determined. Such an analysis is unsatisfactory for the evaluation of a sample which consists of a plurality of virus particles.

Conventionally known attempts to non-destructively analyze virus particles include those which use charge detection mass spectrometry (CDMS) or mass photometry (for example, see Patent Literature 1). By a method described in Patent Literature 1, a virus particle can be non-destructively analyzed to determine the distribution of the detection intensity measured for each mass of the capsid of the plurality of virus particles or to determine whether or not an intended gene has been introduced into each of the plurality of virus particles. However, the stoichiometric proportion of the proteins forming the capsid cannot be determined by those methods.

The problem to be solved by the present invention is to evaluate a sample containing virus particles. More specifically, its objective is to provide a method for evaluating the stoichiometric proportion of one or more kinds of proteins forming the capsid for each virus particle contained in one or more samples.

### Solution to Problem

A method for evaluating a sample containing virus particles according to the present invention developed for solving the previously described problem includes:
the step of preparing a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses;
the step of determining an approximate function which approximates the distribution of the detection intensity of the capsid of the plurality of virus particles detected for each mass of the capsid, the approximate function including the mass as an explanatory variable; and
the step of calculating, for a specified mass which is one of the masses at which a response variable of the approximate function has a predetermined value, the stoichiometry of each of the one or more kinds of proteins forming the capsid having the specified mass, based on the specified mass, the mass of each of the one or more kinds of proteins forming the capsid having the specified mass, and a subunit number which is the total number of the one or more kinds of proteins forming the capsid of the predetermined virus particles.

A device for evaluating a sample containing virus particles according to the present invention developed for solving the previously described problem includes:
a storage unit;
a data input reception unit configured to receive an input of: a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses; the mass of each of the one or more kinds of proteins; and a subunit number which is the total number of the one or more kinds of proteins forming the capsid of the predetermined virus particles;
an approximate function calculation unit configured to determine an approximate function which approximates the distribution of the detection intensity of the capsid of the plurality of virus particles detected for each mass of the capsid, the approximate function including the mass as an explanatory variable;
a specified mass setting unit configured to set, as a specified mass, one of the masses at which a response variable of the approximate function has a predetermined value; and
a stoichiometry calculation unit configured to calculate the stoichiometry of each of the one or more kinds of proteins forming the capsid having the specified mass, based on the specified mass, the mass of each of the one or more kinds of proteins forming the capsid having the specified mass, and the subunit number.

A program for evaluating a sample containing virus particles according to the present invention developed for solving the previously described problem is configured to cause a computer to function as:
a storage unit;
a data input reception unit configured to receive an input of: a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses; the mass of each of the one or more kinds of proteins; and a subunit number which is the total number of the one or more kinds of proteins forming the capsid of the predetermined virus particles;
an approximate function calculation unit configured to determine an approximate function which approximates the distribution of the detection intensity of the capsid of the plurality of virus particles detected for each mass of the capsid, the approximate function including the mass as an explanatory variable;
a specified mass setting unit configured to set, as a specified mass, one of the masses at which a response variable of the approximate function has a predetermined value; and
a stoichiometry calculation unit configured to calculate the stoichiometry of each of the one or more kinds of proteins forming the capsid having the specified mass, based on the specified mass, the mass of each of the one or more kinds of proteins forming the capsid having the specified mass, and the subunit number.

### Advantageous Effects of Invention

The present inventors have conceived the present invention by discovering the fact that the distribution of the detection intensity of a capsid of a plurality of virus particles detected for each mass of the capsid, obtained by an analysis of the plurality of virus particles contained in one or more samples, reflects the heterogeneity of the capsid of the plurality of virus particles in the one or more samples, or in other words, that the difference in the mass of the capsid of the detected virus particles corresponds to the difference in the stoichiometric proportion of the one or more kinds of proteins forming the capsid concerned. According to the present invention, a distribution of a detection intensity of a capsid of a plurality of virus particles detected for each mass of the capsid is prepared, where the distribution is obtained by an analysis of a plurality of predetermined virus particles contained in one or more samples and having a capsid composed of one or more kinds of proteins having known masses. An approximate function, including the mass as an explanatory variable, for approximating that distribution is determined. By this function, a more accurate distribution of the detection intensity of the capsid of the virus particles detected for each mass of the capsid, free from various errors originating from the analyzing device or other factors, can be obtained. Then, for a specified mass which is one of the masses at which a response variable of the approximate function has a predetermined value, the stoichiometry of each of the one or more kinds of proteins forming the capsid having the specified mass is calculated based on the specified mass, the mass of each of the one or more kinds of proteins forming the capsid having the specified mass, and a subunit number which is the total number of the one or more kinds of proteins forming the capsid. Using the calculated result, the stoichiometric proportion of the proteins forming the capsid having the specified mass can be evaluated.

Therefore, according to the present invention, it is possible to evaluate, for each of the virus particles constituting one or more samples, the stoichiometric proportion of the proteins forming the capsid of the virus particle concerned.

### BRIEF DESCRIPTION OF DRAWINGS

FIG. 1 is a diagram showing the main components of a charge detection mass spectrometer according to one embodiment.

FIG. 2 is a diagram showing the main components of an orbitrap according to the embodiment.

FIGS. 3A and 3B are diagrams showing screens for inputting information concerning a virus particle to be analyzed.

FIG. 4 is a diagram illustrating a first calculation process according to the embodiment.

FIG. 5 is a diagram illustrating a second calculation process according to the embodiment.

FIG. 6 is an image showing a result of the first calculation process according to one example.

FIGS. 7A and 7B are tables and graphs showing results of the second calculation process according to one example.

FIG. 8 is a diagram showing the main components of a charge detection mass spectrometer according to one modified example.

FIG. 9 is a diagram showing the main components of an electrostatic linear ion trap according to the modified example.

FIG. 10 is a diagram schematically showing a data processing device according to another modified example.

### DESCRIPTION OF EMBODIMENTS

With reference to the drawings, a charge detection mass spectrometer is hereinafter described which is an illustrative embodiment including a method for evaluating a sample containing virus particles, a device for evaluating a sample containing virus particles and a program for evaluating a sample containing virus particles according to the present invention.

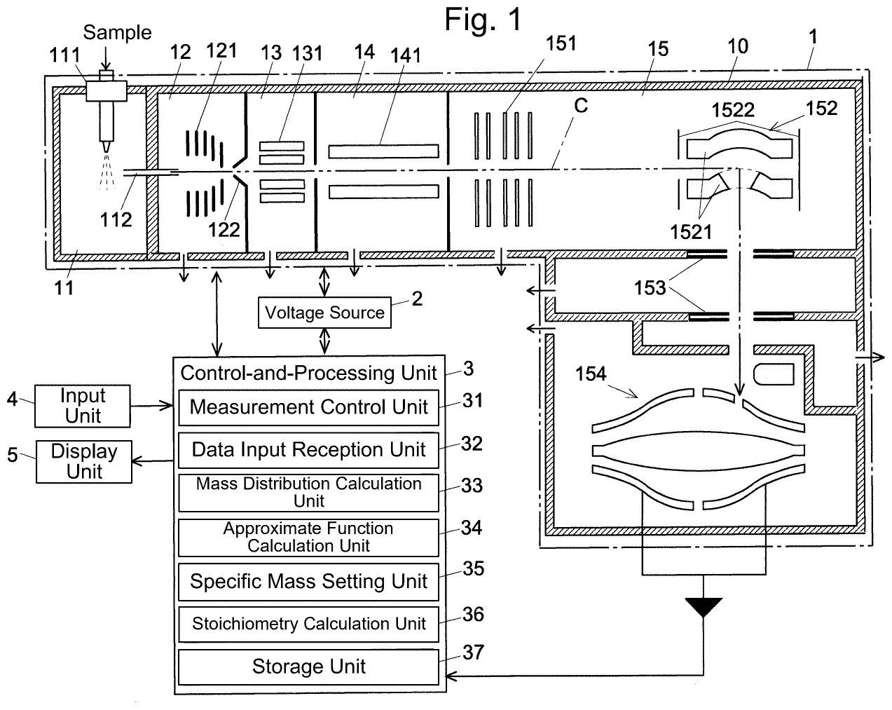

FIG. 1 is a diagram showing the main components of the charge detection mass spectrometer according to the present embodiment. This charge detection mass spectrometer includes a measurement unit 1, voltage source 2, control-and-processing unit 3, input unit 4 and display unit 5.

The measurement unit 1, which is configured to perform a measurement on a sample (liquid sample), includes a vacuum chamber 10 and an ionization chamber 11 connected to the front end of the vacuum chamber 10. The inside of the vacuum chamber 10 is roughly divided into four compartments, i.e., the first vacuum chamber 12, second vacuum chamber 13, third vacuum chamber 14 and fourth vacuum chamber 15. The inside of the ionization chamber 11 is at substantially atmospheric pressure. The degree of vacuum is sequentially increased in a stepwise manner from this ionization chamber 11 through the first vacuum chamber 12, second vacuum chamber 13 and third vacuum chamber 14 to the fourth vacuum chamber 15. That is to say, the configuration of a multi-stage differential pumping system is adopted in the vacuum chamber 10.

The vacuum pumps for evacuating those chambers are omitted in FIG. 1. In general, the first vacuum chamber 12 located next to the ionization chamber 11 is evacuated by a rotary pump, while the subsequent chambers are evacuated by a turbomolecular pump with a rotary pump as a roughing pump.

An electrospray ion (ESI) source 111 is located in the ionization chamber 11. The ionization chamber 11 communicates with the first vacuum chamber 12 through a thin desolvation tube 112. It should be noted that the technique for the ionization is not limited to the ESI method; for example, the matrix-assisted laser desorption/ionization (MALDI) method can also be used. An ion funnel 121 is located within the first vacuum chamber 12. The first and second vacuum chambers 12 and 13 are separated from each other by a skimmer 122 having an opening at its apex. A hexapole ion guide 131 and a quadrupole ion guide 141 are located within the second and third vacuum chambers 13 and 14, respectively. The fourth vacuum chamber 15 contains: ion lenses 151 and 153; a C trap 152 consisting of a curved quadrupole electrode 1521 sandwiched between two endcap lenses 1522, with nitrogen gas introduced inside; and an orbitrap 154.

According to the control signals from the control-and-processing unit 3, the voltage source 2 applies predetermined voltages to the respective electrodes in the related sections of the measurement unit 1, which specifically include electrodes in the ESI source 111, ion funnel 121, hexapole ion guide 131, quadrupole ion guide 141, ion lenses 151 and 153, C trap 152, orbitrap 154 as well as other related sections. Each of the predetermined voltages in the present context is either one of the following types of voltages or a superposition of two or more of them: direct voltage, pulsed voltage, radiofrequency voltage (RF voltage), and alternating voltage having a lower frequency than the RF voltage.

The control-and-processing unit 3 is configured to control the measurement unit 1 directly or via the voltage source 2, as well as to receive and process signals detected in the measurement unit 1. The control-and-processing unit 3 includes, as its functional blocks, a measurement control unit 31, data input reception unit 32, mass distribution calculation unit 33, approximate function calculation unit 34, specified mass setting unit 35, stoichiometry calculation unit 36 and storage unit 37.

Typically, the control-and-processing unit 3 is a personal computer (PC). The functions in the aforementioned functional blocks can be embodied by running, on this PC, a piece of dedicated control-and-processing software installed on the same PC. In that case, the input unit 4 includes a keyboard and a pointing device (e.g., a mouse) provided for the PC, while the display unit 5 is a monitor display provided for the PC.

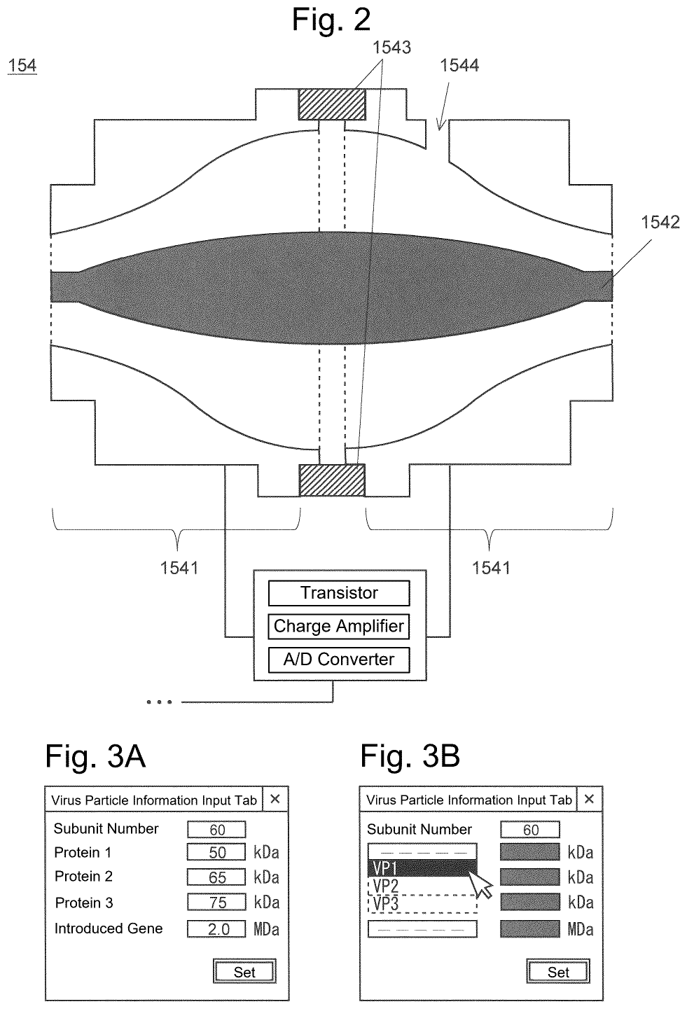

FIG. 2 is a diagram showing the main components of the orbitrap 154 according to the present embodiment. The orbitrap 154 includes the following components: a pair of bowl-shaped electrodes 1541, 1541 each of which has an opening on a central axis; a central electrode 1542 located on the central axis, extending from the opening of one bowl-shaped electrode 1541 to that of the other bowl-shaped electrode 1541; an insulator 1543 configured to create electrical insulation between the two bowl-shaped electrodes 1541, 1541; an ion introduction port 1544 provided in the side wall of one bowl-shaped electrode 1541; as well as a transistor, charge amplifier and analogue-to-digital (A/D) converter housed in a grounded container attached to the outer surface of the outer wall. Although partially omitted in FIG. 2, there are electrical connections between the bowl-shaped electrodes 1541, 1541, transistor, charge amplifier, A/D converter and control-and-processing unit 3. It should be noted that the transistor, charge amplifier and A/D converter (details of which are not shown) are an example of the system for processing signals from the bowl-shaped electrodes 1541, 1541; needless to say, the use and/or combination of these devices can be appropriately changed according to the required performance and/or other conditions. In the charge detection mass spectrometer according to the present embodiment, the orbitrap 154 functions as the detector.

An example of the analytical operation of the charge detection mass spectrometry (CDMS) to be carried out in the charge detection mass spectrometer according to the present embodiment is hereinafter schematically described. A user initially sets a liquid sample containing virus particles to be analyzed (which are hereinafter called the “target virus particles”) into the ESI source 111 and inputs previously obtained information concerning the target virus particles through the input unit 4, such as the total number of one or more kinds of proteins forming the capsid of the target virus particles (“subunit number”), respective masses of the one or more kinds of proteins, mass of the DNA or RNA introduced into the target virus particles, and other related values. These numerical values can be obtained by commonly known analytical techniques. FIGS. 3A and 3B are diagrams showing examples of the screen for inputting information concerning a target virus particle. The input unit 4 and the display unit 5 may be configured to allow the user to directly input the values of the subunit number and the masses, as shown in FIG. 3A, or to allow the user to input the kinds of proteins and gene as shown in FIG. 3B. The data input reception unit 32 receives those inputs and sends the mass value of the DNA or RNA introduced into the target virus particle to the mass distribution calculation unit 33 as well as the subunit number and the mass values of the proteins to the stoichiometry calculation unit 36. In the case where the user has entered the kinds of proteins and gene as shown in FIG. 3B, the data input reception unit 32 refers to a database previously stored in the storage unit 37 for each entered kind of protein or gene to retrieve and send the mass values corresponding to those entered kinds of proteins and gene to the stoichiometry calculation unit 36.

After these tasks have been completed, the user issues a command (input) through the input unit 4 and the display unit 5 to initiate the analysis. The measurement control unit 31 receives the input and controls the voltage source 2 based on the various parameter values stored in the storage unit 37. The voltage source 2 applies the respectively predetermined voltages to the related sections of the measurement unit 1.

The ESI source 111 is supplied with a liquid sample containing the target virus particles. The ESI source 111 electrically charges those virus particles by spraying the supplied liquid sample into the ionization chamber 11 while imparting electric charges to the same sample. The virus particles may be continuously supplied from a sample containing a plurality of virus particles, or alternatively, they may be individually and sequentially supplied from a plurality of samples.

The virus particles electrically charged within the ionization chamber 11, along with electrically charged microdroplets from which the solvent has not sufficiently vaporized, are mainly carried by a gas stream formed by the pressure difference between the pressure within the ionization chamber 11 (which is substantially atmospheric pressure) and the pressure within the first vacuum chamber 12, to be drawn into the desolvation tube 112. The desolvation tube 112 is heated to an appropriate temperature. Therefore, when the electrically charged droplets travel through the desolvation tube 112, the vaporization of the solvent from the droplets is promoted, which helps further charging the virus particles in the liquid sample.

After being ejected from the exit end of the desolvation tube 112 into the first vacuum chamber 12, the virus particles are converged into the vicinity of the ion beam axis C due to the effect of the radiofrequency electric field created by the ion funnel 121. The virus particles converged into the vicinity of the ion beam axis C travel through the opening at the apex of the skimmer 122 and enter the second vacuum chamber 13. Within the second vacuum chamber 13, the solvent and adduct ions which have entered the chamber along with the virus particles are removed, while the virus particles are sent into the third vacuum chamber 14, gaining an amount of kinetic energy corresponding to the magnitude of the direct electric field created by the ion guide 131.

The virus particles which have entered the third vacuum chamber 14 are once more converged into the vicinity of the ion beam axis C due to the effect of the radiofrequency electric field created by the ion guide 141, and enter the fourth vacuum chamber 15.

The virus particles which have entered the fourth vacuum chamber 15 pass through the ion lens 151. The ion lens 151 is configured to adjust the angle and position of incidence of the virus particles into the C trap 152.

The virus particles which have passed through the ion lens 151 enter the C trap 152, to be trapped within the C trap 152 by the radiofrequency voltage applied to the quadrupole 1521 and also by the endcap lens 1522. The nitrogen gas introduced in the C trap 152 cools those particles (which is an operation for making the distribution in kinetic energy of the virus particles as narrow as possible so that the amounts of kinetic energy of those particles are equalized to a predetermined value). The C trap 152 continues collecting virus particles until the amount of collected virus particles reaches a predetermined value. When the predetermined value has been reached, the application of the radiofrequency voltage to the quadrupole 1521 is discontinued, and a direct voltage is applied to the quadrupole 1521 to eject the predetermined amount of virus particles toward the orbitrap 154. In other words, the C trap 152 constantly introduces a specific amount of virus particles into the orbitrap 154 regardless of the concentration of the sample.

The predetermined amount of virus particles ejected from the C trap 152 travel through the plurality of ion lenses 153 and enter the orbitrap 154 from the ion introduction port 1544. The measurement control unit 31 initially increases the voltage of the central electrode 1542 synchronously with the entry of the virus particles into the orbitrap 154 over a typical period of tens of microseconds. The voltage applied to the central electrode 1542 has the opposite sign to the charge of the virus particles to be measured. The virus particles which have entered the orbitrap 154 are further accelerated by the voltage of the central electrode 1542 and begin an orbiting motion around the central electrode 1542. This motion can be expressed by three modes of harmonic oscillations: a rotary motion around the central electrode 1542 (angular frequency ω_{φ}, see the following equation (1)), an oscillation of the radius of orbit of the rotary motion (angular frequency ωᵣ, see the following equation (2)) and a reciprocal motion in the axial direction of the central electrode 1542 (angular frequency ω, see the following equation (3)). In equations (1)-(3), R is the initial radius of orbit at the entry of a virus particle into the orbitrap 154, Rₘ is the current radius of orbit of the virus particle, k is the coefficient of image curvature in the current orbit, and e is the elementary charge. As can be seen in equations (1)-(3), due to the special shape of the orbitrap 154, the only variable included in the angular frequency of the reciprocal motion in the axial direction of the central electrode 1542 is the m/z value of the virus particle. Since the motion of the virus particle induces an electric current in the bowl-shaped electrodes 1541, 1541, information concerning the electric charge and m/z value of the virus particle can be obtained by recording the magnitude and frequency of this current. It should be noted that the rotary motion around the central axis 1542 and the oscillatory motion of the radius of this rotary motion are not to be recorded as signals since the electrodes in which the induced current flows are bowl shaped.

$${ω}_{ϕ} = ω \sqrt{\frac{{( \frac{{R}_{m}}{R} )}^{2} - 1}{2}} \qquad ( 1 )$$

$${ω}_{r} = ω \sqrt{{( \frac{{R}_{m}}{R} )}^{2} - 2} \qquad ( 2 )$$

$$ω = \sqrt{\frac{e}{( m / z )} \cdot k} \qquad ( 3 )$$

After maintaining the orbiting motion of the virus particles around the central electrode 1542 for a predetermined period of time (typically, tens of milliseconds), the measurement control unit 31 adjusts the voltage of the central electrode 1542 so as to eject the virus particles from the opening of the bowl-shaped electrode 1541. The measurement control unit 31 subsequently repeats the previously described operation on the voltage state, causing the orbitrap 154 to repeatedly perform the sequential process of receiving, confining and ejecting the predetermined amount of virus particles, to acquire, for each of the predetermined amount of virus particles, a set of time-series data of the signal detected from the induced current flowing in the bowl-shaped electrodes 1541, 1541.

The transistor, charge amplifier and A/D converter belonging to the orbitrap 154 detect, amplify and discretize the induced current flowing in the bowl-shaped electrodes 1541, 1541 as a signal, respectively, and send it to the control-and-processing unit 3.

The data input reception unit 32 receives, from the measurement unit 1, an input of one set of time-series data of the signal detected from the induced current flowing in the bowl-shaped electrodes 1541, 1541 for each predetermined amount of virus particles and sends that data to the mass distribution calculation unit 33. The mass distribution calculation unit 33 converts each set of time-series data into a frequency spectrum by using an appropriate algorithm, such as the Fast Fourier Transform (FFT). In the frequency spectrum obtained in this manner, a peak appears at the position of the frequency of the reciprocal motion in the axial direction of the central axis 1542 of each virus particle among the predetermined amount of virus particles. The intensity of each peak corresponds to the magnitude of the electric charge of the virus particle which made the reciprocal motion in the axial direction of the central electrode at the frequency corresponding to the peak concerned. For each obtained frequency spectrum, the mass distribution calculation unit 33 reads the intensities of all peaks as well as the values of the frequencies at which those peaks are located. For each frequency at which a peak is located, the mass distribution calculation unit 33 computes, based on the frequency concerned, the m/z value of the virus particle which made the reciprocal motion in the axial direction of the central electrode 1542 at that frequency. Furthermore, the mass distribution calculation unit 33 computes the mass of the virus particle for each peak, based on the m/z value of the virus particle calculated in the aforementioned manner as well as the magnitude of the electric charge of the same virus particle calculated from the intensity of the peak concerned. Ultimately, the mass distribution calculation unit 33 subtracts, from the mass of each virus particle, the value of the mass of the DNA or RNA introduced into that virus particle and creates a distribution of the detection intensity of the capsid of the target virus particles detected for each mass of the capsid. The distribution is sent to the approximate function calculation unit 34.

The distribution of the detection intensity of the capsid of the target virus particles detected for each mass of the capsid, produced by the mass distribution calculation unit 33, normally includes errors originating from the used analyzing device. Accordingly, after receiving the input of the distribution of the detection intensity of the capsid of the target virus particles detected for each mass of the capsid, the approximate function calculation unit 34 determines an approximate function for approximating the distribution, with the mass included as a variable, and sends that function to the specified mass setting unit 35 and the stoichiometry calculation unit 36. The approximate function thus obtained can be considered to be a more accurate expression of the distribution of the detection intensity of the capsid of the virus particles detected for each mass of the capsid from which various errors originating from the analyzing device have been removed. A Gaussian function can typically be used as the approximate function. The approximate function calculation unit 34 can display, on the display unit 5, the outputs from the mass distribution calculation unit 33, i.e., the distribution of the detection intensity of the capsid of the target virus particles detected for each mass of the capsid and the calculated approximate function, in either a superposed or separated form.

Subsequently, the specified mass setting unit 35 sets, as a specified mass or masses, one or more masses at which the value of the response variable of the approximate function is at a predetermined value or within a predetermined range, and sends the mass or masses to the stoichiometry calculation unit 36. The predetermined value or range may be entered by the user through the input unit 4 while visually checking the distribution of the detection intensity of the capsid of the target virus particles detected for each mass of the capsid and/or its approximate function shown on the display unit 5, or a value or range previously stored in the storage unit 37 may be used, or the specified mass setting unit 35 may automatically perform the setting. As for the predetermined value, a local maximum value of the approximate function or one half of the local maximum value may be used, although it is generally possible to use any appropriate value. The predetermined range may be a range equal to or greater than one half of the local maximum value or a range equal to or less than one half of the local maximum value, although it is generally possible to use any appropriate range. In the case of setting, as the specified masses, a plurality of masses at which the values of the response variable of the approximate function fall within a predetermined range, a plurality of mass values may be set as the specified masses at constant or predetermined intervals within that range.

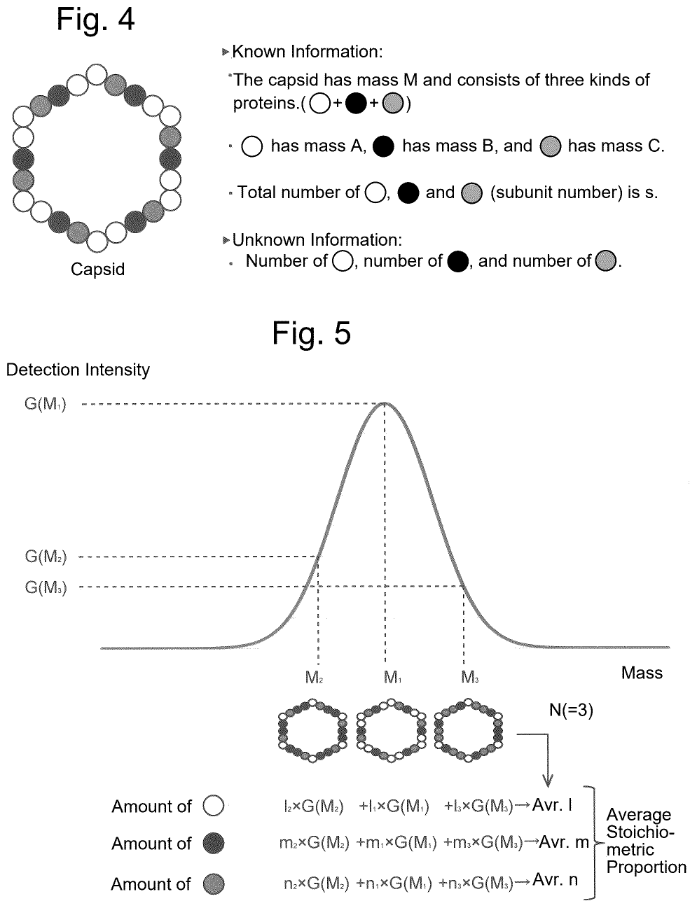

For each of the one or more specified masses received from the specified mass setting unit 35, the stoichiometry calculation unit 36 calculates the stoichiometry (number) of each of the one or more kinds of proteins forming the capsid having the specified mass concerned, based on that specified mass, the mass of each of the one or more kinds of proteins and the subunit number of the capsid concerned. (This calculation is called the “first calculation process” in the present description.) FIG. 4 is a diagram illustrating the first calculation process. Suppose that the capsid of the target virus particles is previously known to be an s-mer (where s is the subunit number) of mass M formed by three kinds of proteins with respective masses A, B and C. The stoichiometry calculation unit 36 calculates three integers 1, m and n which are equal to or greater than zero and satisfy the following equations (4) and (5) as the stoichiometries (numbers) of the three kinds of proteins. It should be noted that these variables cannot be uniquely determined. Therefore, for example, all possible combinations of l, m and n that satisfy the following equations (4) and (5) may be calculated and their average may be selected as a representative solution. Another possibility is to arbitrarily select one reasonably realistic combination of l, m and n as the solution.

$$A \cdot l + B \cdot m + C \cdot n = M \qquad ( 4 )$$

$$l + m + n = s \qquad ( 5 )$$

The present inventors have discovered the fact that the distribution of the detection intensity of a capsid of a plurality of virus particles detected for each mass of the capsid, obtained by an analysis of those virus particles, reflects the heterogeneity of the capsid of the plurality of virus particles, or in other words, that the difference in the mass of the capsid of the detected virus particles corresponds to the difference in the stoichiometric proportion of the one or more kinds of proteins forming the capsid. In the charge detection mass spectrometer according to the present embodiment developed under this concept, the stoichiometry calculation unit 36 calculates the stoichiometry (number) of each of the one or more kinds of proteins forming the capsid having a specified mass, based on the specified mass, the mass of each of the one or more kinds of proteins and the subunit number of the capsid concerned, thereby making it possible to evaluate, for each virus particle contained in a sample, the stoichiometric proportion of the one or more proteins forming the capsid of the particle and assess the properties of the sample containing the virus particle. For example, when the mass at which the response variable of the approximate function has a local maximum value is set as the specified mass, the stoichiometric proportion of the one or more proteins forming the capsid of the virus particle which is most abundantly contained in the sample can be determined. This enables the understanding of representative properties of the sample containing the virus particles. When a plurality of masses at which the values of the response variable of the approximate function are equal to or greater than one half of the local maximum value are set as the specified masses, the stoichiometric proportion of the one or more proteins forming the capsid of a virus particle can be determined for a plurality of virus particles each of which is contained in the sample in a comparatively large quantity, which enables a more exhaustive understanding of the properties of the sample containing the virus particles. Conversely, when a plurality of masses at which the values of the response variable of the approximate function are equal to or less than one half of the local maximum value are set as the specified masses, the stoichiometric proportion of one or more proteins forming the capsid of a virus particle can be determined for a plurality of virus particles each of which is contained in the sample in a comparatively small quantity; in other words, the properties which are rather unlikely for the sample containing the virus particles can be detected and identified. In general, when a mass at which the value of the response variable of the approximate function falls within a predetermined range is set as a specified mass, a plurality of mass values may be set as the specified masses within that range, whereby the properties of the sample containing the virus particles can be evaluated in more detail. In that case, it is preferable to set the plurality of mass values at constant or predetermined intervals as the specified masses.

In the case where a plurality of specified masses are set, the first calculation process can be performed for each of the specified masses; i.e., the stoichiometry (number) can be calculated for each of the one or more kinds of proteins forming the capsid and for each of the specified masses of the capsid. In this case, the stoichiometry calculation unit 36 can calculate the product of the stoichiometry (number) of a kind of protein and the value of the response variable of the approximate function at a specified mass for each of the one or more kinds of proteins forming the capsid and for each of two or more of the specified masses. Based on the total of the product, the stoichiometry calculation unit 36 can calculate an average stoichiometric proportion of the one or more kinds of proteins forming the plurality of capsids corresponding to the two or more specified masses. (This calculation is called the “second calculation process” in the present description.) FIG. 5 is a diagram illustrating the second calculation process. For example, suppose the following conditions: Mᵢ (i=1, 2, . . . , N) are set as the specified masses; for each value of i, a capsid having specified mass Mᵢ is composed of lᵢ proteins of mass A, mᵢ proteins of mass B and nᵢ proteins of mass C; and the value of the response variable of the approximate function at specified mass Mᵢ is G (Mᵢ). Under these conditions, an average stoichiometric proportion l:m:n of the one or more kinds of proteins forming N kinds of capsids can be expressed by one of the following equations (6)-(9). It should be noted that l, m and n in equations (6)-(9) are unrelated to l, m and n in equations (4) and (5) and are not always integers.

$$\begin{matrix}l : m : n & = & {\sum }_{i = 1}^{N} {l}_{i} \cdot G ( {M}_{i} ) : {\sum }_{i = 1}^{N} {m}_{i} \cdot G ( {M}_{i} ) : {\sum }_{i = 1}^{N} {n}_{i} \cdot G ( {M}_{i} ) & ( 6 ) \\ & = & \frac{1}{N} {\sum }_{i = 1}^{N} {l}_{i} \cdot G ( {M}_{i} ) : \frac{1}{N} {\sum }_{i = 1}^{N} {m}_{i} \cdot G ( {M}_{i} ) : \frac{1}{N} {\sum }_{i = 1}^{N} {n}_{i} \cdot G ( {M}_{i} ) & ( 7 ) \\ & = & \begin{matrix}{\sum }_{i = 1}^{N} \frac{{l}_{i}}{{l}_{i} + {m}_{i} + {n}_{i}} \cdot G ( {M}_{i} ) : \\ {\sum }_{i = 1}^{N} \frac{{m}_{i}}{{l}_{i} + {m}_{i} + {n}_{i}} \cdot G ( {M}_{i} ) : {\sum }_{i = 1}^{N} \frac{{n}_{i}}{{l}_{i} + {m}_{i} + {n}_{i}} \cdot G ( {M}_{i} )\end{matrix} & ( 8 ) \\ & = & \begin{matrix}\frac{1}{N} {\sum }_{i = 1}^{N} \frac{{l}_{i}}{{l}_{i} + {m}_{i} + {n}_{i}} \cdot G ( {M}_{i} ) : \\ \frac{1}{N} {\sum }_{i = 1}^{N} \frac{{m}_{i}}{{l}_{i} + {m}_{i} + {n}_{i}} \cdot G ( {M}_{i} ) : \frac{1}{N} {\sum }_{i = 1}^{N} \frac{{n}_{i}}{{l}_{i} + {m}_{i} + {n}_{i}} \cdot G ( {M}_{i} )\end{matrix} & ( 9 )\end{matrix}$$

$$where {I}_{i} + {m}_{i} + {n}_{i} = s for all values of i )$$

In the case where a plurality of specified masses are set, the stoichiometry calculation unit 36 can perform the first calculation process for some or all of the specified masses and subsequently show the calculated results on the display unit 5. In this situation, the stoichiometry calculation unit 36 can allow the user to select one of the one or more kinds of proteins forming the capsid through the input unit 4 and the display unit 5, and sort the displayed results of the first calculation process in ascending or descending order of the stoichiometry of the selected protein. By using this mode of display, the user can efficiently check the stoichiometry of a protein of greater interest (e.g., a protein which affects infectivity) for each virus particle. By this display mode, the stoichiometric proportion of the proteins forming the capsid of a virus particle which will be a virus vector can be recognized as a critical quality attribute for each production lot (culture medium), for example.

### EXAMPLE

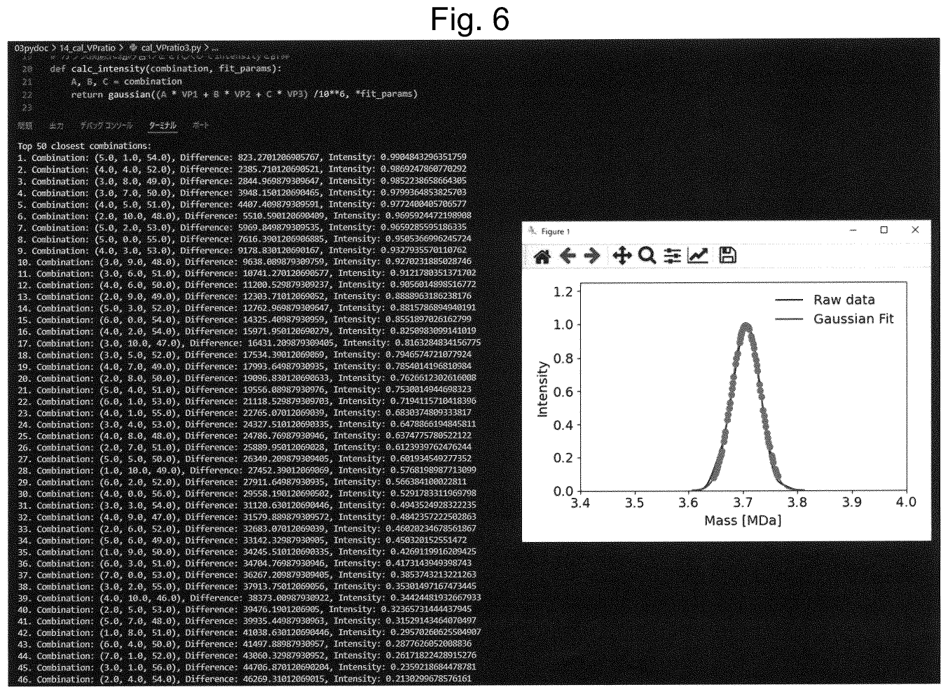

Hereinafter described is an example in which a method for evaluating a sample containing virus particles, a device for evaluating a sample containing virus particles, and a program for evaluating a sample containing virus particles according to the present invention were applied to a sample containing an adeno-associated virus (AAV). FIG. 6 is an image showing a result of the first calculation process according to the present example. On the right panel in FIG. 6 is a graph showing the mass distribution with the mass on the horizontal axis and the intensity on the vertical axis (a distribution of the detection intensity of a capsid of virus particles detected for each mass of the capsid). In this graph, the distribution calculated by the mass distribution calculation unit 33 (“Raw Data”) and an approximate function calculated by the approximate function calculation unit 34 (which is a Gaussian function in the present example; “Gaussian Fit”) are shown in a superposed form. On the left panel in FIG. 6 is a result of the first calculation process. “Combination” in this figure shows the stoichiometric proportion of VP1, VP2 and VP3 which are proteins forming the capsid of AAV, calculated by the first calculation process. The subunit number in the present example is assumed to be 60. “Difference” shows the difference between a mass corresponding to the peak in the graph shown in the right panel in FIG. 6 and the specified mass, with the corresponding position plotted on the graph. “Intensity” shows the intensity at the specified mass concerned. In summary, in the present example, the first calculation process was performed on approximately 50 specified masses within a predetermined section (roughly from 3.6 MDa to 3.8 MDa) around the peak of the mass distribution. From the result shown in FIG. 6, it is possible to clearly understand what kind of virus particle is contained in the sample containing the target virus particles. By this display mode, the stoichiometric proportion of the proteins forming the capsid of a virus particle which will be a virus vector can be recognized as a critical quality attribute for each production lot (culture medium), for example.

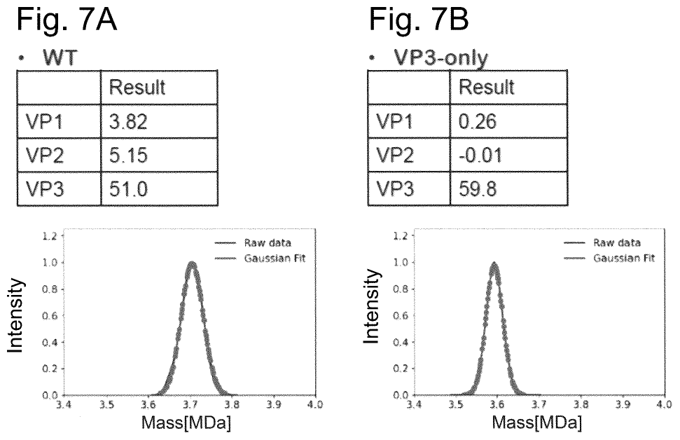

FIGS. 7A and 7B show the results of the second calculation process according to the present example. Specifically, FIG. 7A shows the result obtained for a wild-type strain (WT) of AAV. The second calculation process revealed that the average stoichiometric proportion of the capsid of this type of virus particles was VP1:VP2:VP3=3.82:5.15:51.0. It is generally known that a typical stoichiometric proportion of AAV is VP1:VP2:VP3=5:5:50. The average stoichiometric proportion of AAV contained in the sample according to the present example has a significant difference from the typical stoichiometric proportion. Such a finding concerning a sample containing the target virus particles is useful, for example, in both clinical and research activities for gene therapy. FIG. 7B shows the result obtained for an AAV having a capsid composed of only VP3. The second calculation process revealed that the average stoichiometric proportion of the capsid of this type of virus particles was VP1:VP2:VP3=0.26:−0.01:59.8. This calculation was performed to verify the effect of the present invention by using a virus particle having a capsid whose stoichiometric proportion was previously known. The result shows that the capsid was almost purely composed of VP3. This demonstrates that the present example can correctly evaluate the stoichiometric proportion of the proteins forming a capsid of a virus particle.

### MODIFIED EXAMPLES

The method for evaluating a sample containing virus particles, the device for evaluating a sample containing virus particles, and the program for evaluating a sample containing virus particles according to the present invention are not limited to the previously described embodiment; they can be modified in various forms.

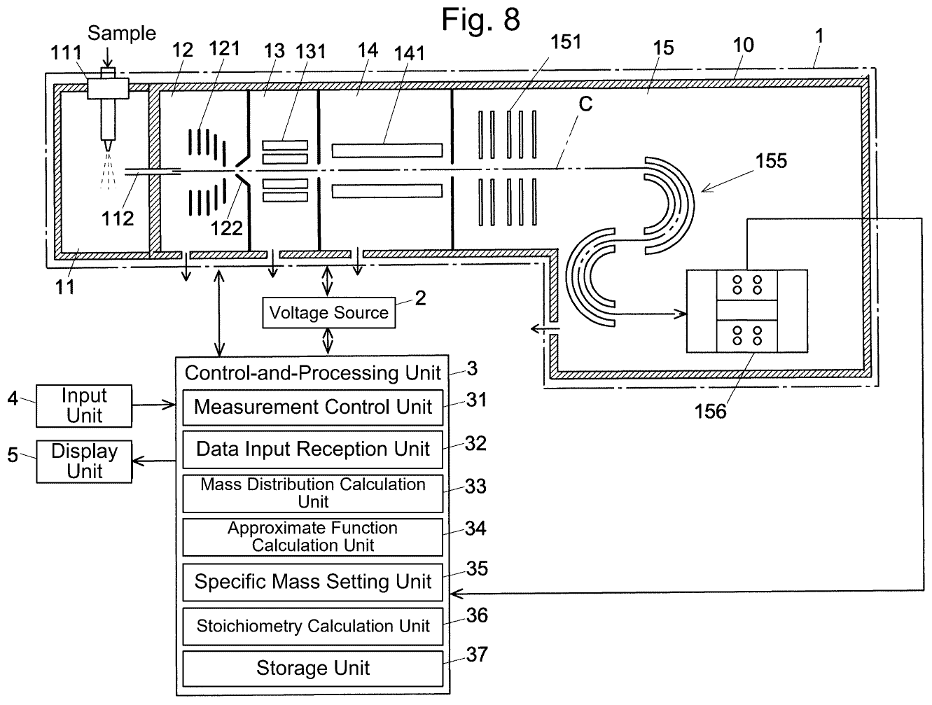

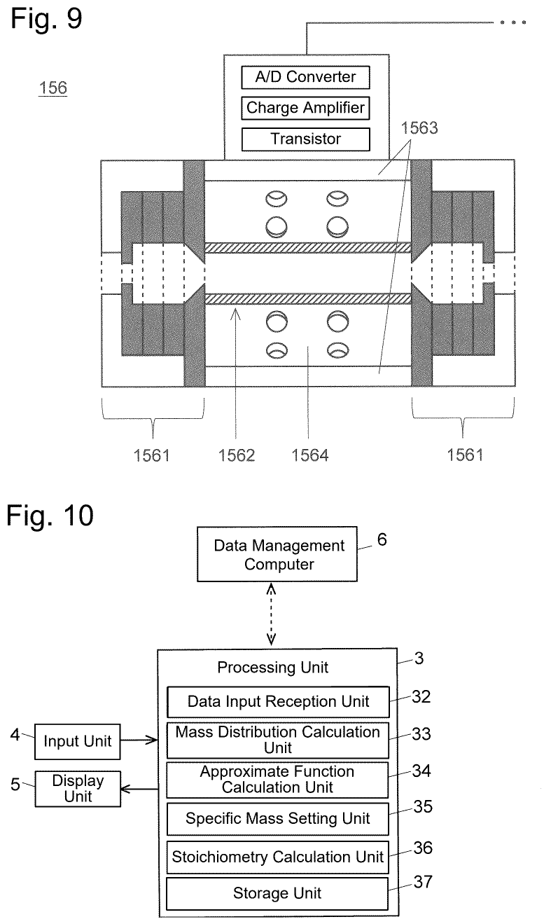

For example, the charge detection mass spectrometer may be an ion trap type of device rather than the orbitrap type as described earlier. FIG. 8 is a diagram showing the main components of an ion trap charge detection mass spectrometer. A difference of the ion trap charge detection mass spectrometer from the orbitrap charge detection mass spectrometer exists in that an ion lens 151, two hemispherical deflection analyzers (HDAs) 155 combined into an S-shaped form (which are hereinafter called the “dual hemispherical deflection analyzer” 155), and an electrostatic linear ion trap 156 are arranged within the fourth vacuum chamber 15. FIG. 9 is a diagram showing the main components of the electrostatic linear ion trap 156 according to the modified example. The electrostatic linear ion trap 156 includes the following components: a pair of endcap electrodes 1561, 1561 each of which has an opening on a central axis; a charge detection tube 1562 located on the central axis in such a manner as to connect the two openings of the endcap electrodes 1561, 1561; an insulator 1564 attached to the inside of an outer wall 1563 which is grounded, holding the charge detection tube 1562; as well as a transistor, charge amplifier and analogue-to-digital (A/D) converter contained in a grounded container attached to the outside of the outer wall 1563. The transistor, charge amplifier and A/D converter may be similarly configured to those of the previously described embodiment. In the charge detection mass spectrometer according to the modified example, the electrostatic linear ion trap 156 functions as the detector.

The operation for the CDMS analysis carried out in the ion trap charge detection mass spectrometer is basically identical to the operation for the CDMS analysis carried out in the orbitrap trap charge detection mass spectrometer until the virus particles are caused to pass through the ion lens 151. In the ion trap charge detection mass spectrometer, the virus particles which have passed through the ion lens 151 travel through the dual hemispherical deflection analyzer 155. As described earlier, the virus particles which have passed through the ion guide 131 due to the effect of the direct electric field created by the ion guide 131 will have an amount of kinetic energy corresponding to the magnitude the direct electric field. Actually, those virus particles may slightly vary in kinetic energy. Accordingly, the dual hemispherical deflection analyzer 155, including two HDAs each of which consists of two concentric electrodes, is configured to create an electric field in the radial direction of the concentric circles, thereby allowing only virus particles having a predetermined amount of kinetic energy. In other words, the dual hemispherical deflection analyzer 155 allows only virus particles having that predetermined amount of kinetic energy to enter the electrostatic linear ion trap 156. The reason for combining two HDAs to construct the S-shaped dual hemispherical deflection analyzer 155 is to maximally reduce the variation in kinetic energy of the virus particles entering the electrostatic linear ion trap 156. The present configuration also helps the virus particles to maintain their original direction of travel.

The virus particles which have passed through the dual hemispherical deflection analyzer 155 enter the electrostatic linear ion trap 156. The measurement control unit 31 initially sets the voltage of the endcap electrode 1561 through which the virus particles enter (this electrode is hereinafter called the “front endcap electrode 1561A”) to zero and the voltage of the other endcap electrode 1561 (which is hereinafter called the “rear endcap electrode 1561B”) to a predetermined value (this state of voltage is hereinafter called the “first voltage state”), thereby allowing a single virus particle to pass through the opening in the front endcap electrode 1561A and enter the charge detection tube 1562.

After maintaining the first voltage state for a predetermined period of time (typically, tens of microseconds), the measurement control unit 31 changes the voltage of the front endcap electrode 1561A to a predetermined value while maintaining the voltage of the rear endcap electrode 1561B (this state of voltage is hereinafter called the “second voltage state”) whereby the virus particle which has entered the electrostatic linear ion trap 156 is confined within the charge detection tube 1562. That is to say, the virus particle is repelled by the endcap electrodes 1561, 1561 and makes a reciprocal motion within the charge detection tube 1562.

After maintaining the second voltage state for a predetermined period of time (typically, tens of milliseconds), the measurement control unit 31 changes the voltage of the rear endcap electrode 1561B to zero while maintaining the voltage of the front endcap electrode 1561A (this state of voltage is hereinafter called the “third voltage state”), whereby the virus particle is ejected from the opening in the rear endcap electrode 1561B.

After maintaining the third voltage state for a predetermined period of time (typically, a few to several milliseconds), the measurement control unit 31 changes the voltage state of the endcap electrodes 1561, 1561 to the first voltage state. The measurement control unit 31 subsequently repeats the previously described operation of the voltage state, causing the electrostatic linear ion trap 156 to successively perform the sequential process of receiving, confining and ejecting a single virus particle.

In the electrostatic linear ion trap 156, when an electrically charged virus particle passes through the inner space of the charge detection tube 1562, a virtual charge (image charge) which is opposite in sign and identical in magnitude to the charge of the virus particle is induced on the surface of the charge detection tube 1562. The transistor, charge amplifier and A/D converter belonging to the electrostatic linear ion trap 156 detect, amplify and discretize the magnitude of the virtual charge induced on the surface of the charge detection tube 1562 as a signal, respectively, and send it to the control-and-processing unit 3. Such a signal is detected every time the virus particle moves through the inner space of the charge detection tube 1562 while the voltage state of the endcap electrodes 1561, 1561 is maintained in the second voltage state (the virus particle is confined). By repeating the operation of causing the voltage state of the endcap electrodes 1561, 1561 to cyclically transition through the first, second and third voltage states at predetermined intervals of time, the measurement control unit 31 acquires, for each virus particle, time-series data of a signal corresponding to the magnitude of the virtual charge induced on the surface of the charge detection tube 1562.

The data input reception unit 32 receives an input of the time-series data of the signal corresponding to the magnitude of the virtual charge induced on the surface of the charge detection tube 1562, acquired for each virus particle in the measurement unit 1, and sends the data to the mass distribution calculation unit 33. The mass distribution calculation unit 33 converts each set of time-series data into a frequency spectrum by using an appropriate algorithm, such as the Fast Fourier Transform (FFT). In the frequency spectrum obtained in this manner, a high peak appears at each of the fundamental and overtone frequencies of the reciprocal motion of the virus particle within the charge detection tube 1562 (these frequencies are hereinafter simply called the “fundamental frequency” and the “overtone frequency”, respectively) corresponding to that frequency spectrum (alternatively, a configuration in which only the fundamental frequency appears is also possible, as in the case of the so-called Gen6Trap). The intensity of the peak which appears at the fundamental frequency corresponds to the magnitude of the charge of the virus particle corresponding to the frequency spectrum concerned. The mass distribution calculation unit 33 reads, from each of the acquired frequency spectra, the value of the fundamental frequency, i.e., the value of the lowest frequency among the frequencies which are integral multiples of the fundamental frequency, and the intensity of the peak located at the position of the fundamental frequency. The mass distribution calculation unit 33 also reads various parameter values stored in the storage unit 37 and calculates the kinetic energy of each virus particle from the values of the voltages applied to the ion guide 131 and the dual hemispherical deflection analyzer 155. Then, for each virus particle, the mass distribution calculation unit 33 calculates the m/z value of the virus particle based on the value of the fundamental frequency and the value of the kinetic energy of the virus particle. Furthermore, for each virus particle, the mass distribution calculation unit 33 calculates the mass of the virus particle based on the m/z value of the virus particle and the magnitude of the charge of the virus particle calculated by detecting the virtual charge induced on the charge detection tube 1562. Ultimately, the mass distribution calculation unit 33 subtracts, from the mass of each virus particle, the value of the mass of the DNA or RNA introduced into that virus particle and creates a distribution of the detection intensity of the capsid of the target virus particles detected for each mass of the capsid. The distribution is sent to the approximate function calculation unit 34. Since the subsequent processing is identical to the previously described embodiment, its description will be omitted.

In the present invention, the distribution of the detection intensity of the capsid of the virus particles detected for each mass of the capsid does not always need to be prepared by CDMS; for example, it may also be prepared by mass photometry. Mass photometry is a method in which the molecular weight of a biological molecule (e.g. a nucleic acid, protein, AAV vector or aggregate) or nanoparticle in a sample is measured from the intensity of the scattered light from the sample placed on a glass substrate. For example, this method can be realized by changing the configuration of the measurement unit 1, voltage source 2 and other related sections.

The present invention does not need to be used with an analyzing device in an integrated form. For example, as shown in FIG. 10, the present invention can also be realized as a data processing device including a data management computer 6 and a processing unit 30.

As can be understood from the comparison of FIGS. 7A and 7B, when the average stoichiometric proportion of the proteins forming the capsid is completely different, the position at which the peak emerges in the mass distribution may possibly be different. Furthermore, when a virus particle with a specific gene introduced and one without that specific gene are mixed in the same sample, a plurality of peaks may possibly be recognized in the mass distribution. The mass distribution calculation unit 33 or the approximate function calculation unit 34 may be configured so that, when a plurality of peaks (local maximum values) are recognized in the distribution of the detection intensity of a capsid of virus particles detected for each mass of the capsid, the unit 33 or 34 extracts subsets of data, with each subset including one of those peaks, and sends them to the specified mass setting unit 35, stoichiometry calculation unit 36, storage unit 37 and display unit 5. Furthermore, the mass distribution calculation unit 33 or the approximate function calculation unit 34 may also be configured so that, when the difference between the masses corresponding to two peaks is roughly equal to the mass of the introduced gene (e.g., within an error range of +5%), the unit 33 or 34 shows, on the display unit 5, a piece of information explaining the situation separate from or superposed on the distribution of the detection intensity of the capsid of the target virus particles detected for each mass of the capsid or the approximate function of that distribution.

### [Modes]

It is evident to a person skilled in the art that the previously described illustrative embodiment is a specific example of the following modes of the present invention.
(Clause 1) A method for evaluating a sample containing virus particles according to one mode of the present invention includes:
the step of preparing a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses;
the step of determining an approximate function which approximates the distribution of the detection intensity of the capsid of the plurality of virus particles detected for each mass of the capsid, the approximate function including the mass as an explanatory variable; and
the step of calculating, for a specified mass which is one of the masses at which a response variable of the approximate function has a predetermined value, the stoichiometry of each of the one or more kinds of proteins forming the capsid having the specified mass, based on the specified mass, the mass of each of the one or more kinds of proteins forming the capsid having the specified mass, and a subunit number which is the total number of the one or more kinds of proteins forming the capsid of the predetermined virus particles.
(Clause 2) A device for evaluating a sample containing virus particles according to one mode of the present invention includes:
a storage unit;
a data input reception unit configured to receive an input of: a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses; the mass of each of the one or more kinds of proteins; and a subunit number which is the total number of the one or more kinds of proteins forming the capsid of the predetermined virus particles;
an approximate function calculation unit configured to determine an approximate function which approximates the distribution of the detection intensity of the capsid of the plurality of virus particles detected for each mass of the capsid, the approximate function including the mass as an explanatory variable;
a specified mass setting unit configured to set, as a specified mass, one of the masses at which a response variable of the approximate function has a predetermined value; and
a stoichiometry calculation unit configured to calculate the stoichiometry of each of the one or more kinds of proteins forming the capsid having the specified mass, based on the specified mass, the mass of each of the one or more kinds of proteins forming the capsid having the specified mass, and the subunit number.
(Clause 7) A program for evaluating a sample containing virus particles according to one mode of the present invention is configured to cause a computer to function as:
a storage unit;
a data input reception unit configured to receive an input of: a distribution of a detection intensity of a capsid of plurality of virus particles detected for each mass of the capsid, the distribution obtained by an analysis of a plurality of predetermined virus particles contained in one or more samples and having a capsid composed of one or more kinds of proteins having known masses; the mass of each of the one or more kinds of proteins; and a subunit number which is the total number of the one or more kinds of proteins forming the capsid of the predetermined virus particles;
an approximate function calculation unit configured to determine an approximate function which approximates the distribution of the detection intensity of the capsid of the plurality of virus particles detected for each mass of the capsid, the approximate function including the mass as an explanatory variable;
a specified mass setting unit configured to set, as a specified mass, one of the masses at which a response variable of the approximate function has a predetermined value; and
a stoichiometry calculation unit configured to calculate the stoichiometry of each of the one or more kinds of proteins forming the capsid having the specified mass, based on the specified mass, the mass of each of the one or more kinds of proteins forming the capsid having the specified mass, and the subunit number.

By the method for evaluating a sample containing virus particles according to Clause 1, the device for evaluating a sample containing virus particles according to Clause 2 and the program for evaluating a sample containing virus particles according to Clause 7, a distribution of a detection intensity of a capsid of a plurality of virus particles detected for each mass of the capsid is prepared, where the distribution is obtained by an analysis of a plurality of predetermined virus particles contained in one or more samples and having a capsid composed of one or more kinds of proteins having known masses. An approximate function, including the mass as an explanatory variable, for approximating that distribution is determined. By this function, a more accurate distribution of the detection intensity of the capsid of the virus particles detected for each mass of the capsid, free from various errors originating from the analyzing device or other factors, can be obtained. Then, for a specified mass which is one of the masses at which a response variable of the approximate function has a predetermined value, the stoichiometry of each of the one or more kinds of proteins forming the capsid having the specified mass is calculated based on the specified mass, the mass of each of the one or more kinds of proteins forming the capsid having the specified mass, and a subunit number which is the total number of the one or more kinds of proteins forming the capsid. Using the calculated result, the stoichiometric proportion of the proteins forming the capsid having the specified mass can be evaluated.
(Clause 3) In a device for evaluating a sample containing virus particles according to Clause 3, which is a device for evaluating a sample containing virus particles according to Clause 2, the specified mass setting unit is configured to set a plurality of masses as the specified mass, and the stoichiometry calculation unit is configured to calculate, for each of the plurality of specified masses, the stoichiometry of each of the one or more kinds of proteins forming the capsid having the specified mass concerned, based on the specified mass, the mass of each of the one or more kinds of proteins forming the capsid having the specified mass, and the subunit number.

By the device for evaluating a sample containing virus particles according to Clause 3, the properties of a sample containing virus particles can be evaluated in more detail by setting a plurality of mass values as the specified masses.
(Clause 4) In a device for evaluating a sample containing virus particles according to Clause 4, which is a device for evaluating a sample containing virus particles according to Clause 3, the stoichiometry calculation unit is configured to calculate, for each of the plurality of specified masses, the stoichiometry of each of the one or more kinds of proteins forming the capsid having the specified mass concerned, based on the specified mass, the mass of each of the one or more kinds of proteins forming the capsid having the specified mass, and the subunit number, and to subsequently calculate, for two or more of the plurality of specified masses and for each of the one or more kinds of proteins, the product of the stoichiometry of the protein forming the capsid having the specified mass concerned and the value of the response variable of the approximate function at the specified mass concerned, and to calculate the total of the product.

The device for evaluating a sample containing virus particles according to Clause 4 calculates, for two or more of the plurality of specified masses and for each of the one or more kinds of proteins, the product of the stoichiometry of the protein forming the capsid having the specified mass concerned and the value of the response variable of the approximate function at the specified mass concerned, and calculates the total of the product. This enables the calculation of an average stoichiometric proportion of the one or more kinds of proteins forming a plurality of capsids corresponding to the plurality of specified masses.
(Clause 5) In a device for evaluating a sample containing virus particles according to Clause 5, which is a device for evaluating a sample containing virus particles according to one of Clauses 2-4, the specified mass setting unit is configured to set, as the specified mass, a mass at which the response variable of the approximate function has a local maximum value.

The device for evaluating a sample containing virus particles according to Clause 5 can determine the stoichiometric proportion of the one or more kinds of proteins forming the capsid of a virus particle which is most abundantly contained in a sample. This enables the understanding of the representative properties of a sample containing virus particles.
(Clause 6) In a device for evaluating a sample containing virus particles according to Clause 6, which is a device for evaluating a sample containing virus particles according to one of Clauses 3-5, the specified mass setting unit is configured to set, as the specified masses, a plurality of masses at each of which the value of the response variable of the approximate function is equal to or greater than a predetermined value.

By the device for evaluating a sample containing virus particles according to Clause 6, the stoichiometric proportion of one or more proteins forming the capsid of a virus particle can be determined for a plurality of virus particles each of which is contained in the sample in a comparatively large quantity. This enables a more exhaustive understanding of the properties of the sample containing the virus particles.

### REFERENCE SIGNS LIST

1 . . . . Measurement Unit
10 . . . . Vacuum Chamber
11 . . . . Ionization Chamber
111 . . . . Electrospray Ion (ESI) Source
112 . . . . Desolvation Tube
12 . . . . First Vacuum Chamber
121 . . . . Ion Funnel
122 . . . . Skimmer
13 . . . . Second Vacuum Chamber
131, 141 . . . . Ion Guide
14 . . . . Third Vacuum Chamber
15 . . . . Fourth Vacuum Chamber
151, 153 . . . . Ion Lens
152 . . . . C Trap
1521 . . . . Quadrupole Electrode
1522 . . . . Endcap Lens
154 . . . . Orbitrap
1541 . . . . Bowl-Shaped Electrode
1542 . . . . Central Electrode
1543 . . . . Insulator
1544 . . . . Ion Introduction Port
155 . . . . Dual Hemispherical Deflection Analyzer
156 . . . . Electrostatic Linear Ion Trap
1561 . . . . Endcap Electrode
1562 . . . . Charge Detection Tube
1563 . . . . Outer Wall
1564 . . . . Insulator
2 . . . . Voltage Source
3 . . . . Control-and-Processing Unit
30 . . . . Processing Unit
31 . . . . Measurement Control Unit
32 . . . . Data Input Reception Unit
33 . . . . Mass Distribution Calculation Unit
34 . . . . Approximate Function Calculation Unit
35 . . . . Specified Mass Setting Unit
36 . . . . Stoichiometry Calculation Unit
37 . . . . Storage Unit
4 . . . . Input Unit
5 . . . . Display Unit
6 . . . . Data Management Computer

1. A method for evaluating a sample containing virus, comprising:
a step of preparing a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses;
a step of determining an approximate function which approximates the distribution of the detection intensity of the capsid of the plurality of virus particles detected for each mass of the capsid, the approximate function including the mass as an explanatory variable; and
a step of calculating, for a specified mass which is one of the masses at which a response variable of the approximate function has a predetermined value, a stoichiometry of each of the one or more kinds of proteins forming the capsid having the specified mass, based on the specified mass, the mass of each of the one or more kinds of proteins forming the capsid having the specified mass, and a subunit number which is a total number of the one or more kinds of proteins forming the capsid of the predetermined virus particles.

2. A device for evaluating a sample containing virus particles, comprising:
a storage unit;
a data input reception unit configured to receive an input of: a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses; the mass of each of the one or more kinds of proteins; and a subunit number which is a total number of the one or more kinds of proteins forming the capsid of the predetermined virus particles;
an approximate function calculation unit configured to determine an approximate function which approximates the distribution of the detection intensity of the capsid of the plurality of virus particles detected for each mass of the capsid, the approximate function including the mass as an explanatory variable;
a specified mass setting unit configured to set, as a specified mass, one of the masses at which a response variable of the approximate function has a predetermined value; and
a stoichiometry calculation unit configured to calculate a stoichiometry of each of the one or more kinds of proteins forming the capsid having the specified mass, based on the specified mass, the mass of each of the one or more kinds of proteins forming the capsid having the specified mass, and the subunit number.

3. The device for evaluating a sample containing virus particles according to claim 2, wherein:
the specified mass setting unit is configured to set a plurality of masses as the specified mass; and
the stoichiometry calculation unit is configured to calculate, for each of the plurality of specified masses, the stoichiometry of each of the one or more kinds of proteins forming the capsid having the specified mass concerned, based on the specified mass, the mass of each of the one or more kinds of proteins forming the capsid having the specified mass, and the subunit number.

4. The device for evaluating a sample containing virus particles according to claim 3, wherein the stoichiometry calculation unit is configured to calculate, for each of the plurality of specified masses, the stoichiometry of each of the one or more kinds of proteins forming the capsid having the specified mass concerned, based on the specified mass, the mass of each of the one or more kinds of proteins forming the capsid having the specified mass, and the subunit number, and to subsequently calculate, for two or more of the plurality of specified masses and for each of the one or more kinds of proteins, a product of the stoichiometry of the protein forming the capsid having the specified mass concerned and a value of the response variable of the approximate function at the specified mass concerned, and to calculate a total of the product.

5. The device for evaluating a sample containing virus particles according to claim 2, wherein the specified mass setting unit is configured to set, as the specified mass, a mass at which the response variable of the approximate function has a local maximum value.

6. The device for evaluating a sample containing virus particles according to claim 3, wherein the specified mass setting unit is configured to set, as the specified masses, a plurality of masses at each of which a value of the response variable of the approximate function is equal to or greater than a predetermined value.

7. A non-volatile storage medium recording a program for evaluating a sample containing virus particles, wherein the program is configured to cause a computer to function as:
a storage unit;
a data input reception unit configured to receive an input of: a distribution of a detection intensity of a capsid of a plurality of virus particles detected for each mass of the capsid, the distribution obtained by an analysis of a plurality of predetermined virus particles contained in one or more samples and having a capsid composed of one or more kinds of proteins having known masses; the mass of each of the one or more kinds of proteins; and a subunit number which is a total number of the one or more kinds of proteins forming the capsid of the predetermined virus particles;
an approximate function calculation unit configured to determine an approximate function which approximates the distribution of the detection intensity of the capsid of the plurality of virus particles detected for each mass of the capsid, the approximate function including the mass as an explanatory variable;
a specified mass setting unit configured to set, as a specified mass, one of the masses at which a response variable of the approximate function has a predetermined value; and
a stoichiometry calculation unit configured to calculate a stoichiometry of each of the one or more kinds of proteins forming the capsid having the specified mass, based on the specified mass, the mass of each of the one or more kinds of proteins forming the capsid having the specified mass, and the subunit number.
